(19) United States
(12) Patent Application Publication
(10) Pub. No.: US 2013/0190611 A1
(43) Pub. Date: Jul. 25, 2013

(54) METHOD FOR DETERMINING A DEVIATION OF A MEDICAL INSTRUMENT FROM A TARGET POSITION

(71) Applicant: Siemens Aktiengesellschaft, Munchen (DE)

(72) Inventor: RAINER GRAUMANN, HOECHSTADT (DE)

(73) Assignee: SIEMENS AKTIENGESELLSCHAFT, MUNCHEN (DE)

(21) Appl. No.: 13/747,661

(22) Filed: Jan. 23, 2013

(30) Foreign Application Priority Data
Jan. 23, 2012 (DE) 10 2012 200 921.3

(51) Int. Cl. A61B 5/06; A61B 6/12; A61B 5/00

(52) U.S. Cl. CPC A61B 5/061; A61B 5/0059; A61B 5/7475; A61B 6/12
USPC 600/424

(57) ABSTRACT

In a method for determining a deviation of a medical instrument from a target position, in an image of an implant located in the body of a patient created with an imaging system, a virtual model of the implant is fitted at the correct location and thus a location of the implant is determined in the imaging system. On a basis of the location of the virtual model in the imaging system and a target position for an instrument selected as a fixed position relative to the virtual model, the location of the target position in the imaging system is determined. A video image of the instrument is created by a recording device and its current location in relation to the recording device is determined from this. By a geometrical relationship between the recording device and the imaging system, a deviation of the instrument from the target position is determined.

### CROSS-REFERENCE TO RELATED APPLICATION

This application claims the priority, under 35 U.S.C. §119, of German application DE 10 2012 200 921.3, filed Jan. 23, 2012; the prior application is herewith incorporated by reference in its entirety.

### BACKGROUND OF THE INVENTION

### Field of the Invention

The invention relates to a method for determining a deviation of a medical instrument from a target position.

Implants, which must be fixed to the individual bone fragments to be treated, are used in surgical treatment of fractures of a bone for example, so that the bones can heal in a desired way relative to one another. The implants are fixed to the individual bone fragments with a suitable attachment device, for example screws. In accordance with the anatomy of the bone and the geometry of the implants, these fixings must fulfill specific geometrical requirements. For screws in particular the requirements are their length and the angle at which they have to be introduced into the implant or the bone respectively. These types of geometrical parameter can be determined intraoperatively by a planning method and by planning tools suitable for the method.

However the implementation of the planning data obtained by the planning method is problematic, i.e. turning the required values into reality, for example maintaining the planned angle when inserting a screw into the implant. One is dependent here on the implementation skills of the medical personnel. To facilitate the implementation what are referred to as drilling sleeves are used, which restrict the angular range too approximately +/−20% in the coordinate system of the implant. The angular range however only represents an external framework for implementing the planning data. It is thus still down to the skill of the medical personnel to adhere to the precise required value. Depending on the experience of the medical personnel, there are therefore greater deviations from the required value.

### SUMMARY OF THE INVENTION

It is accordingly an object of the invention to provide a method for determining a deviation of a medical instrument from a target position which overcomes the above-mentioned disadvantages of the prior art methods of this general type, with which medical personnel are supported during the above-mentioned medical measure so that there can be better adherence to a required value.

Accordingly the method for determining a deviation of a medical instrument from a target position includes the now describes steps.

In a first step a) a virtual model of the implant is fitted at the correct location into a picture generated by an imaging system of the implant located in a body of a patient and thus its location in the imaging system is determined.

In step b) on the basis of the location of the virtual model in the imaging system and a target position for an instrument selected as fixed relative to the virtual model, the location of the target position in the imaging system is determined.

In step c) a video image of the instrument is created by a recording device and its location in relation to the recording device is determined from this image.

In step d) a deviation is finally determined by use of a known geometrical relationship between the recording device and the imaging system.

Fitting the virtual model of the implant into the generated image at the correct location device that first of all the location of the model in the coordinate system of the imaging system is known. The target position for an instrument, for example a tool such as a screwdriver, is viewed as a position into which the instrument must be moved in order to fulfill a geometrical requirement. For example it can be the position in which the instrument engages with an attachment device for the implant such that the attachment device assumes a desired location. Since the target position for the instrument is initially known merely in relation to the implant, i.e. in the coordinate system of the implant, in step b), on the basis of this information and the location of the virtual model in the imaging system determined in step a), the location of the target position in the imaging system can be determined. The fact that the target position is selected as a fixed position relative to the virtual model, that the target position, although it is initially for example able to be selected as any given position on the basis of the anatomical circumstances and a selection of an attachment device for the implant, is fixed however after its selection in relation to the implant, i.e. has uniquely-defined coordinates in the coordinate system of the implant. Thus after step b) both the location of the virtual model of the implant and also the location of the target position in the imaging system is known.

In step c) a video image of the instrument is then generated by the recording device. To this end the instrument is equipped with optical markers, so that the instrument can be registered in the video image. This enables its current location in relation to the recording device to be determined. Since the video image is refreshed at a specific frequency the respective current position can thus also be repeatedly refreshed during a procedure in an operation.

Since the geometrical relationship is also known between the imaging system and the recording device for the video image, a deviation of the instrument from the target position can then be determined in step d). In other words: the relationship between the coordinate system of the imaging system and the coordinate system of the recording device is known, so that coordinates of the virtual model in the imaging system determined in step a) and coordinates of the location of the target position in the imaging system determined in step b) can be converted in each case into coordinates of the coordinate system of the recording device. Thus both the coordinates of the virtual model and also of the target position in the coordinate system of the recording device are known. Since the coordinates of the instrument in relation to the recording device have also been determined, the deviation from the target position can be determined from this. On the basis of this deviation the instrument can then be correctly positioned.

It is thus possible in this fashion to position an instrument exactly and thus for example to insert screws for implant fixing at a specific angle in the implant. A significant advantage of the method is that no additional x-ray radiation is necessary for it, since the instrument is positioned entirely under optical image control.

Preferably a correction parameter is determined on the basis of the deviation, which is visualized by a user interface. This can be done for example on the basis of a crosshair or arrows which point in the appropriate direction.

In a preferred version of the invention the location of the target position is selected as a permanent location relative to the virtual model in that the location is planned on the basis of the image created with the imaging system and of the virtual model fitted into the image. For this purpose, as is known from the prior art, the suitable attachment device, for example a screw with a specific diameter and an optimal length can be planned initially. Thereafter the angle is selected and thus defined at which the attachment device must be inserted into the implant.

Usually an x-ray system is used as the imaging system in step a). i.e. the image created is available as a 2D x-ray projection image and thus serves as a basis for fitting a model of the implant available in 3D form for example, which can be undertaken by a known 2D/3D registration. This type of fitting into the image can also be done with the planning tool.

If the video image is created with a camera augmented mobile c-arm (CAMC) system, the coordinates of the imaging system correspond to the coordinates of the recording device. Therefore in step d) a transformation of the coordinates of the imaging system into the coordinates of the imaging device is no longer required, so that therefore in step a) the location of the virtual model of the implant and in step b) the location of the target position are already known in the coordinate system of the recording device. This makes a deviation of the instrument from the target position able to be determined even more easily.

In a further preferred embodiment of the invention, a number of fixed target positions lying on a guide curve are selected. It is guaranteed in this fashion that an attachment device, such as a screw for example, can be inserted on a desired path by the instrument into the implant or the bone. In particular the guide curve describes a straight line for insertion of a screw.

In order to make a further improvement of the guidance of the instrument possible, as well as the current position of the instrument provided with an optical marker, the virtual model of the implant and the target position are incorporated into the video image at their correct location. Thus a user can easily detect whether there is a deviation of an actual position of the instrument from the required position, i.e. the target position.

The properties, features and advantages of this invention described above, as well as the manner in which these are achieved will become clearer and easier to understand in conjunction with the description of exemplary embodiments given below, which are explained in greater detail in conjunction with the drawings.

Other features which are considered as characteristic for the invention are set forth in the appended claims.

Although the invention is illustrated and described herein as embodied in a method for determining a deviation of a medical instrument from a target position, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration of an imaging system and a recording device for creating a video image of an implant located in a body of a patient according to the invention;

FIG. 2 is an illustration of an image created with the imaging system;

FIG. 3 is an illustration of a virtual model of an implant;

FIG. 4 is an illustration of a video image created with the recording device; and

FIG. 5 is an illustration of a correction parameter presented by a monitor.

### DETAILED DESCRIPTION OF THE INVENTION

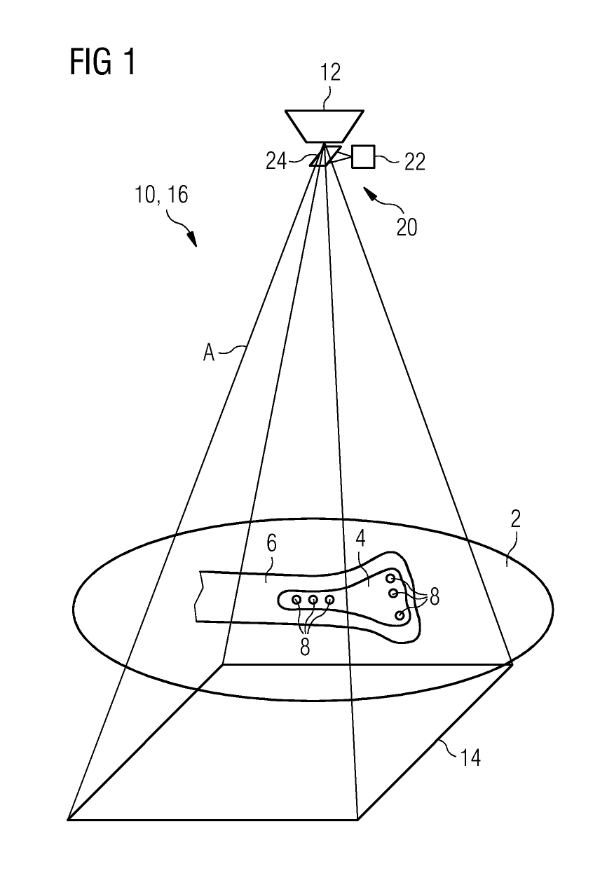

FIG. 1 shows an initial situation for carrying out the inventive method. As part of an operation an implant 4 has been inserted in the body of a patient, which in the exemplary embodiment is used for treatment of a bone 6. To fix the implant 4 to the bone 6, the implant has a number of holes 8 through which a suitable attachment device, for example screws, can be inserted into the bone 6. To guarantee a sufficient hold of the implant 6 on the bone, the attachment device must have a specific length and be introduced into the implant or into the bone 6 respectively at a specific angle. The inventive method is carried out in order to guarantee the latter procedure with sufficient accuracy.

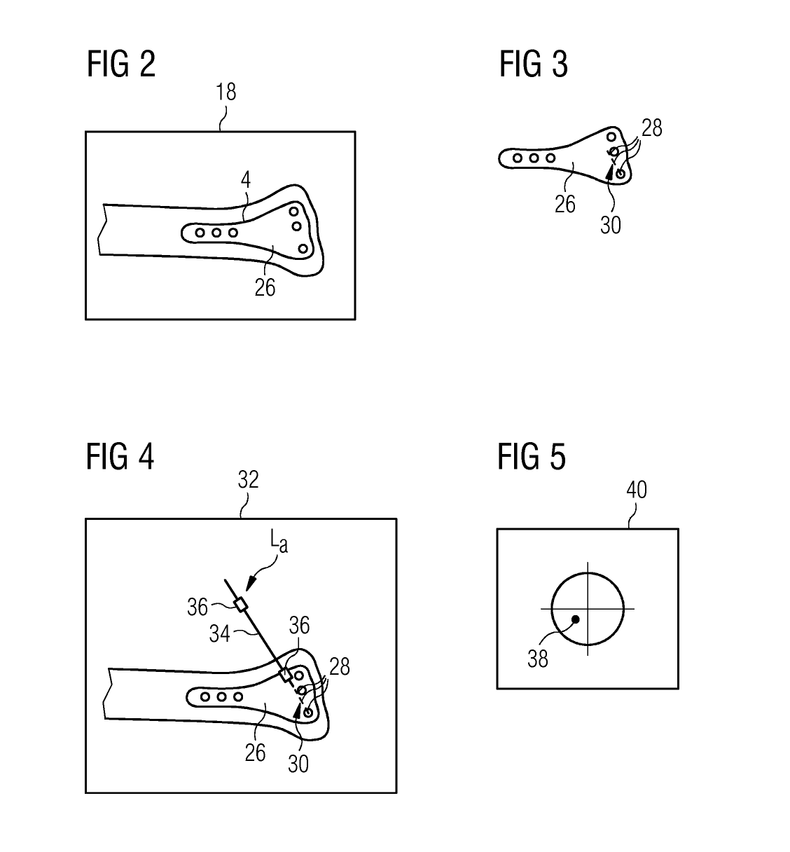

In this method, in a step a) an image, which is depicted in FIG. 2, is created by an imaging system 10, which is formed by an x-ray system 16 containing an x-ray source 12 and an x-ray detector 14. The X-ray source 12 and the x-ray detector 14 are for example fixed to a C-arm not shown in FIG. 1. Also shown in FIG. 1 is a recording device 20 which is formed from a video camera 22 and a mirror 24. The recording device 20 in this case is likewise fixed to the C-arm not shown in the figure and has the same recording geometry A as the x-ray system 16. This means that the coordinate system of the recording device 20 and the imaging system 10 are identical. Thus an object located in the recording geometry A has the same coordinates both in the coordinate system of the imaging system 10 and also in the coordinate system of the recording device 20.

In step a) of the inventive method a virtual model 26 of the implant 4, which is available in three-dimensional form, is fitted by a 2D/3D registration method known from the prior art at the correct location into the image 18, as is shown in FIG. 2. This means the virtual model 26 is moved virtually on the basis of the information of the image 18 so that it has the same location as the real implant 4 has in the imaging system 10. On the basis of this location of the virtual model 26 after its fitting into the image 18 and the known recording geometry A of the imaging system, its location in the imaging system 10 can thus be determined.

Thereafter, on the basis of the virtual model 26 of the implant 4, a target position 28 is selected as a fixed position in relation to the virtual model 26, as is shown in FIG. 3. This thus means that the target position 28 is determined in the coordinate system of the implant 4. The target position 28 is initially freely selectable and is determined on the basis of the geometry of the implant 4 and of the bone 6 and is then fixed in relation to the implant 4 and thus of its coordinate system. Such a definition can for example be made in advance by selecting a specific attachment device which can only be inserted at a pre-specified angle into the implant. The determination can however also be planned on the basis of the image created with the imaging system 10 and the virtual model 26 fitted into the image intraoperatively on the basis of a planning tool. By the planning tool both lengths of the attachment devices needed and also the angle at which they must be inserted into the implant are determined, i.e. selected as fixed values. The results of the planning are then required values of a guide curve 30 along which the attachment devices must be inserted into the implant 4 or the bone 6. These required values correspond to the target positions 28, so that in this case a number of target positions 28 lying on a guide curve 30 are planned. In this case the guide curve 30 describes a straight line since the attachment devices must be inserted along the straight line into the implant 4 or the bone 6 respectively. However all other curve variants are possible on the basis of which the attachment devices are to be inserted into the implant 4.

In step b) the location of the target position 28 in the imaging system 10 is now determined on the basis the location of the virtual model 26 in the imaging system 10 and the target position 28 is selected as a fixed position relative to the virtual model 26, in this case planned.

FIG. 4 now shows a video image 32 of a medical instrument 34, here a screwdriver, created with the recording device 20. The instrument has two optical markers 36 to enable it to be detected in the video image 32, so that in step c) of the inventive method, on the basis of the video image 32, a current location Lₐ of the instrument 34 in relation to the recording device 20 can be determined. Since a CAMC system serves as the recording device 20 in the exemplary embodiment, which means that the recording geometry A of the imaging system 10 and recording device 20 are identical, after the determination of the respective coordinates of the virtual model 26 and the target position 28 in the imaging position 10, their coordinates in relation to the recording device 20 are thus also automatically fixed at the same time.

In step d), through a known geometrical relationship, i.e. here the identity between the coordinate systems of the imaging system 10 and of the recording device 20, a deviation of the actual location Lₐ of the instrument 34 from the target position 28 can be determined.

On the basis of the deviation a correction parameter 38 can then be determined, which is visualized by a user interface 40 for example on a monitor (FIG. 5). This correction parameter 38 can for example be represented by a crosshair. It would however also be possible to represent the correction parameter 38 in the form of arrows, which specify the direction in which the instrument 34 is to be moved.

Thus a user, supported by the inventive method, can now guide the medical instrument 34, for example a screwdriver for screwing the screws into the implant 4 or into the bone 6, into the determined target position 28. The fact that the number of fixed selected target positions 28 represent a guide curve 30 forming a straight line enables the instrument 34 to be guided using the inventive method from one target position 28 into the next target position so that the attachment device to be inserted into the implant 4 are inserted at the fixed angle selected, i.e. in this case at the planned angle. For further improvement of the guidance of the instrument 30 the video image 32 can also be included, into which the virtual model 26 of the implant 4 and the target positions 28 are also incorporated, as is shown in FIG. 4. In addition models of the attachment device and further significant geometrical parameters, such as a longitudinal axis of an attachment device and its extent for example, could also be incorporated into the video image 32.

With the presence of a CAMC functionality in particular, a very simple method for exact implementation of the planning results for correct introduction of the attachment device is produced. A significant advantage is that no additional x-ray radiation is required for this. The instrument is aligned entirely under optical image control.

Although the invention has been illustrated and described in greater detail by the preferred exemplary embodiment, the invention is not restricted by the disclosed examples and other variations can be derived therefrom by the person skilled in the art, without departing from the scope of protection of the invention.

### LIST OF REFERENCE CHARACTERS

2 Body
4 Implant
6 Bone
8 Hole
10 Imaging system
12 X-ray source
14 X-ray detector
16 X-ray system
18 Image
20 Recording device
22 Video camera
24 Mirror
26 Model
28 Target position
30 Guide curve
32 Video image
34 Instrument
36 Marker
38 Correction parameter
40 User interface
A Recording geometry
Lₐ Current location

1. A method for determining a deviation of a medical instrument from a target position, which comprises the steps of:
a) fitting, in an image of an implant disposed in a body of a patient created with an imaging system, a virtual model of the implant at a correct location and thus a location of the implant is determined in the imaging system;
b) determining a further location of the target position in the imaging system on a basis of the correct location of the virtual model in the imaging system and the target position for the medical instrument selected as a fixed position relative to the virtual model;
c) creating a video image of the medical instrument by a recording device and determining a current location of the medical instrument in relation to the recording device from the video image; and
d) determining a deviation of the medical instrument from the target position by means of a known geometrical relationship between the recording device and the imaging system.

2. The method according to claim 1, which further comprises, on a basis of the deviation, determining a correction parameter, which is visualized by means of a user interface.

3. The method according to claim 1, which further comprises selecting the further location of the target position as a fixed location relative to the virtual model, in that this is planned on a basis of the image created with the imaging system and the virtual model fitted into the image.

4. The method according to claim 1, which further comprises providing in the step a) an x-ray system as the imaging system.

5. The method according to claim 1, which further comprises creating the video image with a camera augmented mobile c-arm system.

6. The method according to claim 1, which further comprises selecting a number of target positions on a guide curve as fixed positions.

7. The method according to claim 6, wherein the guide curve describes a straight line.

8. The method according to claim 1, which further comprises incorporating the virtual model of the implant and the target position at the correct location into the video image.
